(19) United States
(12) Patent Application Publication
(10) Pub. No.: US 2008/0282733 A1
(43) Pub. Date: Nov. 20, 2008

(54) MEMORY GEMSTONES

(76) Inventors: Ravi TOLWANI, New York, NY (US); Liza Burns, Castle Rock, CO (US)

Correspondence Address: GLENN PATENT GROUP, 3475 EDISON WAY, SUITE L, MENLO PARK, CA, 94025, US

(21) Appl. No.: 12/108,463

(22) Filed: Apr. 23, 2008

Related U.S. Application Data
(60) Provisional application No. 60/913,514, filed on Apr. 23, 2007.

(51) Int. Cl. A44C 17/00; C12P 7/00; C12P 5/02

(52) U.S. Cl. 63/32; 435/132; 435/167

(57) ABSTRACT

Animal or human tissue is digested to extract carbon-containing gases. The digestion process can include hydrolysis, acidogenesis, acetogenesis, and methanogenesis. The step of denitrification can be added if too much nitrogen is produced during digestion. Digestion can also include enzymes that break down the tissue in preparation for methanogenesis. The carbon-containing gas can be added onto or be incorporated throughout a natural or synthetic diamond or other gemstone. The carbon-containing gas can be added during hydrothermal synthesis to create a gemstone with the carbon-containing gas incorporated throughout.

### PRIORITY CLAIM AND RELATED APPLICATIONS

This patent application claims the benefit of provisional application Ser. No. 60/913,514, titled “Pet Hair Memory Diamonds,” filed on Apr. 23, 2007, the entirety of which is hereby incorporated by reference.

### FIELD OF THE INVENTION

Embodiments of the invention relate generally to the field of gemstones, and more specifically to gemstones created by incorporating organic and inorganic molecules derived from animal and human tissues.

### BACKGROUND

Human life can be characterized as a series of interactions. Thus, when we lose someone essential to our lives, the loss can be devastating. Because we are sentimental creatures, carrying a memento of another person or pet can help ease suffering and provide comfort. Thus, having a memento that contains a part of that person or animal would be of great value.

A gemstone is a mineral, rock, or petrified material that when faceted, i.e. cut, and polished is a collectible or can be used in jewelry. Gemstones consist of, but are not limited to, diamonds, rubies, emeralds, sapphires, amethyst, opals, aquamarine, tanzanite, and alexandrite. Gems are classified into different groups, species, and varieties. For example, ruby is the red variety of the species corundum, while any other color of corundum is considered sapphire. Emerald (green), aquamarine (blue), bixbite (red), goshenite (colorless), heliodor (yellow), and morganite (pink) are all varieties of the mineral species beryl.

Some gemstones are manufactured to imitate other gemstones. For example, cubic zirconia is a synthetic diamond simulant composed of zirconium oxide. The imitations copy the look and color of the real stone but possess neither their chemical nor physical characteristics. True synthetic gemstones, however, are not imitations. For example, diamonds, ruby, sapphires, and emeralds have been manufactured in labs. These gems possess very nearly identical chemical and physical characteristics to the naturally occurring variety.

Synthetic corundums, including ruby and sapphire, are very commonly manufactured. Synthetic gems are produced by both solution and melt crystallization techniques. Flame fusion, also called the Verneuil process, is a common method of manufacturing synthetic gemstones. It is primarily used to produce the ruby and sapphire varieties of corundum, as well as the diamond simulants rutile and strontium titanate. A finely powdered substance is melted with an oxyhydrogen flame and the melted droplets form a crystal known as a boule. In the case of manufacturing rubies or sapphires, the powdered substance is alumina. Crystals produced by the flame fusion process are chemically and physically equivalent to their naturally occurring counterparts.

Other processes can also be used to grow gem-quality synthetic corundum. These processes include, but are not limited to, flux-growth and hydrothermal synthesis. In the marketplace, flame-fusion and crystal-pulled synthetics (corundum and spinel) are relatively less expensive and therefore more abundant than the flux and hydrothermal synthetics. Synthetic diamonds can be grown from a metallic flux at high temperatures and pressures by chemical vapor deposition (CVD).

Diamond growth has been achieved by CVD using gaseous carbon compounds such as hydrocarbons or carbon monoxide (See Eversole W G, U.S. Pat. Nos. 3,030,187 and 3,030,188, Kamo et al 1983). CVD using low pressure techniques, such as microwave plasma-assisted CVD (MPCVD) can produce high quality diamond epitaxial films (See Sato and Kamo, 1992). Epitaxial growth occurs when the diamond has the same crystalline orientation as the substrate upon which it is grown. When high temperatures were used, MPCVD resulted in growth of pure diamond films, both epitaxially and non-epitaxially, at very high growth rates (See Vohra et al. U.S. Pat. No. 5,628,824). High growth rates were also observed for homoepitaxial synthesis of diamond at low pressures (See Snail and Hanssen, 1991). In addition to diamond films, high rate homoepitaxial growth of diamonds to produce single crystal diamonds has been accomplished under different conditions, including high temperature in an atmospheric-pressure flame.

The “plasma” referred to in MPCVD is a completely ionized gas which contains equal numbers of ions and electrons. Although it is envisioned that the most useful way of generating plasma is by microwave radiation, other standard techniques for generating plasmas that are typically used for CVD may also be employed, for example, by application of direct current or radio frequency radiation, or employing a DC plasma jet or arc. Another method provides for the formation of a plasma ball which encompasses the diamond substrate. This is primarily due to the placement of the substrate on a molybdenum screw which facilitates the breakdown of the diamond-forming gas into plasma by its metallic composition and sharp edges. However, other techniques for generating plasmas should also be operable if care is taken to maintain appropriate temperature of the substrate to avoid thermal runaway.

A gemstone can be produced with carbon from human or animal tissues. The carbon is derived from keratin, which is a fibrous structural protein. The carbon can be extracted through the process of anaerobic digestion and fermentation. Thus, what is needed is an efficient, repeatable process for converting human and animal tissue into a carbon-containing gas, which is then deposited onto a gemstone to create a memory gem.

### SUMMARY OF THE INVENTION

Animal, human, or processed tissues are incorporated into natural or synthetic gems to produce memory gems. One embodiment of the present invention relates generally to producing a carbon-containing gas, such as methane, from fermentation or by other non-fermentative processes, from tissues or tissue products. Byproducts of tissues include, but are not limited to, ash (following incineration of tissues), protein (following proteinase processing of tissues), DNA, or keratin (such as keratin as present in hair or feathers). Proteinase processing involves the protein digestion of tissues in reagents such as proteinase K (available from Sigma). Standard molecular biology techniques are used to extract DNA or RNA from tissues. In one embodiment, the carbon-containing gas is an intermediate.

The carbon-containing gas is used to create a single crystal diamond, coat an existing (natural or synthetic) diamond, or coat an existing non-diamond gem (natural or synthetic) to produce memory gems. Tissues and tissue products, usually in smaller amounts, are added to reagents for production of synthetic gems. For example, during flame-fusion and hydrothermal synthesis, small amounts of tissue are incorporated into synthetic gems to produce memory gems.

In another embodiment, the tissues, and their organic and/or inorganic derivatives, are more directly incorporated into synthetic gems to produce memory gems. For example, animal and human tissues, or its organic and inorganic processed derivates, such as, for example, but not limited, to, ash, are converted into synthetic gems.

The features and advantages described in this summary and the following detailed description are not all-inclusive, and particularly, many additional features and advantages will be apparent to one of ordinary skill in the art in view of the drawings, specification, and claims hereof. Moreover, it should be noted that the language used in the specification has been principally selected for readability and instructional purposes, and may not have been selected to delineate or circumscribe the inventive subject matter, resort to the claims being necessary to determine such inventive subject matter.

### BRIEF DESCRIPTION OF THE DRAWINGS

FIG. 1 is a diagram illustrating the production of a memory gem from tissue by creating carbon-containing gas according to one embodiment of the present invention.

FIG. 2 is a diagram illustrating the production of carbon-containing gas from tissue using four different embodiments of the present invention.

FIG. 3 is a diagram illustrating the digestion process to convert organic reagents into methane and carbon dioxide according to another embodiment of the present invention.

FIG. 4 is a diagram illustrating various ways to produce memory gems from carbon-containing gas according to several embodiments of the present invention.

The Figures depict embodiments of the present invention for purposes of illustration only. One skilled in the art will readily recognize from the following discussion that alternative embodiments of the structures illustrated herein may be employed without departing from the principles of the invention detailed herein.

### DETAILED DESCRIPTION

### Definitions

carbon-containing gases: hydrocarbon gases, especially acetylene, ethylene, propane and methane.

diamond: a transparent crystal of tetrahedral bonded carbon atoms. Contains exceptional physical characteristics such as extreme hardness, a high dispersion index, and high thermal conductivity.

diamond-forming gas: compounds that are generally employed in the art of chemical vapor deposition (CVD) for the purposes of depositing diamond onto substrates.

incorporation: direct or indirect insertion, processed insertion or inclusion.

inorganic tissues: tissues or cells, or derivatives of tissues or cells, with very little or no detectable carbon molecules. Includes, but not limited to, carbon-containing tissues that were ‘processed’ to remove all carbon molecules.

methanogenesis: the formation of methane by a group of microbes known as methanogens.

methanogenic bacteria: bacteria that consume various substrates, most commonly acetic acid, and produce methane gas along with other byproducts, most commonly carbon dioxide.

organic tissue: tissue or cells, or derivatives of tissues or cells, that contain carbon molecules.

processed tissues: tissues processed by, but not limited to, digestion (proteinases, etc.), chemicals (e.g. denature, etc), and fermentation (e.g. methanogenesis).

synthetic gems: gems that are created in the laboratory to mimic natural gems.

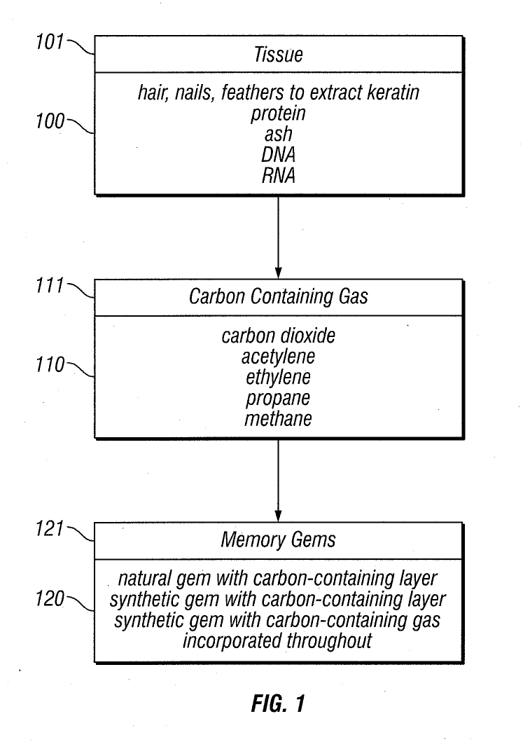

The production of memory gems has two phases: I. conversion of tissues to a carbon-containing gas and II. using the carbon-containing gas to produce a memory gem. FIG. 1 illustrates an embodiment of the overall framework. Tissue 101 can include, for example, hair, nails, and feathers for keratin, protein, ash, DNA and RNA. Tissue 101 is used to produce carbon-containing gases 111. These gases can include, for example, carbon dioxide, acetylene, ethylene, propane, and methane. Carbon-containing gases 111 are used to produce memory gems 121. In one embodiment, the memory gems 121 are produced by adding a layer of carbon-containing gas 111 to a natural or synthetic gem. In another embodiment, the carbon-containing gas 111 is incorporated throughout the memory gem 121.

I. Conversion of Tissues and Processed Tissues from Animals and Humans to a Carbon-Containing Gas

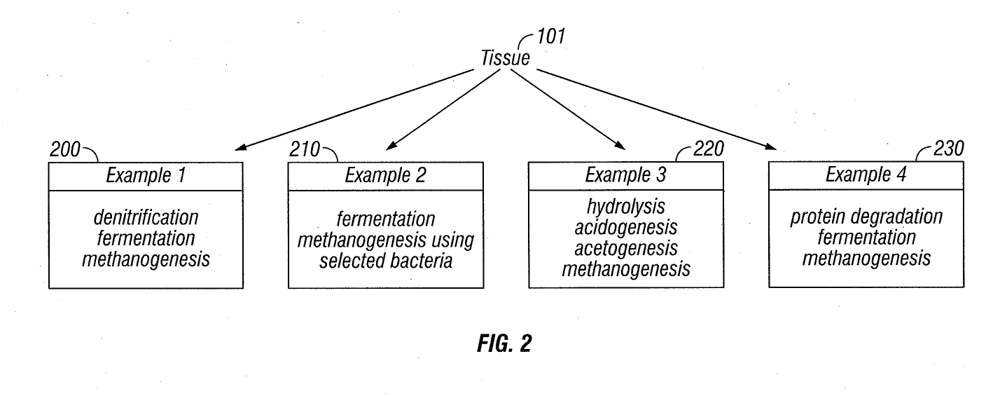

Examples of embodiments of the present invention are illustrated in FIG. 2.

### EXAMPLE 1

### Fermentation and Methanogenesis Using Methanogenic Consortia to Convert Animal and Plant Tissues and Tissue Products, to Carbon-Containing Gas

Example 1 200 involves the steps of denitrification, fermentation, and methanogenesis using an anaerobic methanogenic consortia. Fermentation is the breakdown of chemicals into alcohols and acids in an anaerobic environment. One type of fermentation step is hydrolysis. Next, hydrolysis products are combined with methanogens to produce carbon-containing gas, such as, but not limited to, methane and carbon dioxide. The following is a specific protocol.

Specific Protocol to Produce Methane from Hair, Keratin and Other Tissues.

Extracting carbon from hair and other tissues rich in keratin is a challenge because the tissues degrade poorly under anaerobic conditions. Thus, before starting methanogenesis, denitrification and hydrolysis pretreatment is typically performed. These processes are not always necessary. In addition, as discussed in Example 3, a four-part digestion process of hydrolysis, acidogeniesis, acetogenesis, and methanogenesis, may also be used

### Step 1: Denitrification—Ammonia Stripping

When tissue substrates, such as hair, contain high amounts of nitrogen molecules, ammonia is a common byproduct of chemical reactions (See Koster 1989). A high ammonia concentration reduces the efficiency of methanogenesis (See Angelidaki and Ahring, 1993), resulting in less methane. In order to minimize the inhibitory effects of ammonia production on methanogenesis, an ammonia stripping, or denitrification step, is conducted.

Reagent (hair, nails, etc.) is placed in media as described below. Ammonia stripping requires a temperature of over 85° C. degrees and a pH greater than 10.5. Under these conditions, the ammonia produced is removed as ammonia gas. The ammonia gas is removed via air stripping, which is the process of forcing air through the solution, to leave solubilized, denitrified substrates (See Laio et al 1995). In preparation for methanogenesis, a buffer is used to neutralize the pH of the media to 7.0.

### Step 2: Hydrolysis

Hydrolysis is a chemical reaction where the high keratin/high protein rich substrates (hair, nails) are broken down into more soluble and fermentation-friendly byproducts including sugars, amino acids, and fatty acids. The enzymes used to digest the keratin include, but are not limited to keratinase, collagenase, elastase, endopeptidase, and/or proteinase. In addition, certain proteolytic bacteria, for example Clostridium species, can be used.

Alternatively, the two pre-treatments can be combined. This can be accomplished by concurrent enzymatic treatment (commercial alkaline endopeptidase, 1-10 g/l) at high temperature (120° C., 5 minutes).

### Step 3: Methanogenesis

A methanogenic consortia, that is, a mixture of methanogens, is typically used for anaerobic digestion. Methanogens of genus Methanosarcina spp. and Methanosaeta spp. can be used. Specific examples of methogens include Methanosarcina barkeri and Methanosaeta concilli. Other bacteria may be used, such as hydrogen-oxydizing methanogens. Bacterial strains include Methanobacterium ruminantium, M. formicium, M thermoautotrophicum, M arbophilicum, M barkeri. Consortia may be obtained from anaerobic waste degradation plants.

Denitrified and hydrolyzed reagents are added to sterile media prepared for the culture of the methogenic bacteria. Media is prepared to consist of nutrients and trace metal solutions as reported by Speece, 1996.

Nutrient/Trace Metal Solution Preparation (See Speece, 1996)

| Compound | g/L of solution |
|---|---|
| NH4Cl | 12.608 |
| KH2PO4 | 0.439 |
| Na2S2O3-5H2O | 2.554 |
| CaCl2-2H2O | 0.477 |
| MgCl2-6H2O | 0.151 |
| FeCl2-4H2O | 0.0819 |
| NiCl2-6H2O | 0.0162 |
| CoCl2-6H2O | 0.0121 |
| ZnCl2 | 0.0417 |

In order to optimize methanogenesis, a vitamin source is added to the medium. Anywhere from 1× to 4× vitamins of vitamin source below may be added to optimize methanogenesis.

TABLE
1X Vitamin Solution Formulation

| Component | Amount (mg/L) |
|---|---|
| Biotin | 2.0 |
| Folic Acid | 2.0 |
| Pyridoxine hydrochloride | 10.0 |
| Thiamine-HCl | 5.0 |
| Riboflavin | 5.0 |
| Nicotinic Acid | 5.0 |
| Calcium D-(+)-pantothenate | 5.0 |
| Vitamin B12 | 0.1 |
| p-Aminobenzoic acid | 5.0 |
| Thioctic acid | 5.0 |

For experiments, small scale methane fermentation will be carried out using 40 ml sealed serum vials containing 20 ml of medium (or other size vials where medium volume is approximately 50% of entire vial volume, allowing sufficient head space for gas collection). Media will be maintained at pH of 7.0 at 35° C. to 40° C. (for mesophilic methanogens) or at higher temperatures (55° C.) for thermophilic range methanogens.

Experiments are maintained in anaerobic conditions. Experiments may, for example, be conducted in a Coy Products (Grass Lake, Mich.) anaerobic flexible vacuum chamber. The vacuum chamber headspace will contain a 95% nitrogen-5% hydrogen gas mixture.

Gas produced, including methane and carbon dioxide, is pulled occasionally from the serum vials using the rubber stopper and analyzed by gas chromatography for methane content. Methane produced will be stored in gas bags or other containers, such as 50 ml conical tubes. Up to 1 L of gas is collected for subsequent analysis. In certain cases, gas bags will be used to capture the methane produced. To capture the gas, gas is extracted close to the head space of the vial. Once some methane is produced, methane withdrawal may occur repeatedly, with methane removed using a syringe by inserting into stopper of sealed serum vials. Repeated draws may occur to collect gas produced.

As an alternative from sealed serum vials, a 3 neck flask (ACE glass, New Jersey) can be used for methanogenesis stage as described (Taconi K A, 2004). Here, one neck of the flask is used for measuring pressure, another neck for extracting gas. This system allows for continual monitoring of gas pressure and extraction of gases produced over time.

During the reaction some ammonia may be produced necessitating ammonia stripping. While the prior denitrification step should minimize this problem, repeated ammonia stripping may be needed to increase methanogenesis efficiency. In addition to ammonia inhibition of methanogenesis, hydrogen sulfide production can also be inhibitory. If hydrogen sulfide is produced, steps will be taken to neutralize, in order to maintain efficient methanogenesis.

### EXAMPLE 2

### Fermentation Using Specific Methanogenic Bacteria, Either Engineered or Naturally Isolated, to Convert Tissues to Carbon-Containing Gas

As illustrated in FIG. 2, Example 2 210 is similar to the process outlined above in Example 1 200, but instead of using a methanogenic consortia, a specific methanogenic bacterial strain or two will be used. Specific conditions for growth and methanogenesis, such as pH and temperature variations, may vary and depends upon the methanogenic bacterial strain employed.

### EXAMPLE 3

### Use of Four-Stage Digestion Process for Converting Tissues to Carbon-Containing Gas

The above two examples use fermentation and methanogenesis. Example 3 220, as illustrated in FIG. 2, describes digestion. Anaerobic digestion is a biological process where organic matter is degraded to methane in an anaerobic environment. Digestion occurs in four steps: hydrolysis, acidogenesis, acetogenesis, and methanogenesis.

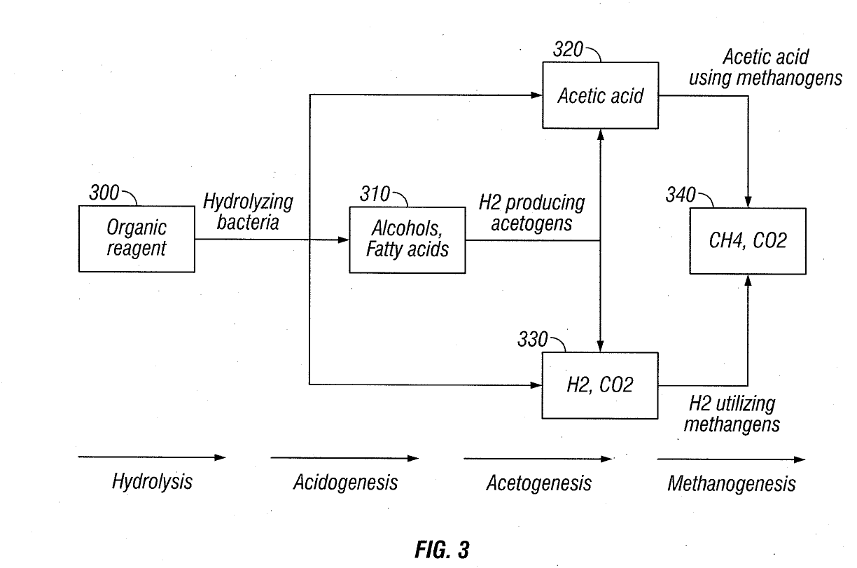

The four stages of digestion are illustrated in FIG. 3. Hydrolysis occurs when the organic reagent 300 is combined with acid-forming bacteria. Acid forming bacteria can be obtained from a variety of mechanisms, including heat treating anaerobic inoculums to deactivate methanogens and retain the acidogens. The bacteria break the keratin down into simple monomers. Next, during acidogenesis the monomers are broken down into alcohols 310 and fatty acids 310. During acetogenesis, bacteria known as acetogens convert the fatty acids 310 into acetic acid 320 with hydrogen 330 and carbon dioxide 330 as byproducts. Lastly, methanogenesis is the process of using methanogens to consume the hydrogen 330 and convert the acetic acid 320 into methane 340 and carbon dioxide 340.

### EXAMPLE 4

### Use of a Protein Degradation Process to Convert Animal and Human Tissues to a Protein Substrate for Conversion to Carbon-Containing Gas

Example 4 230 is illustrated in FIG. 2. In certain cases, tissues are processed by molecular biology reagents prior to carbon gas production. Processing can occur by enzymatic reaction. As an example, it is envisioned that tissues and their products will be predigested enzymatically with molecular biology reagents, or enzymes, such as proteinases, to derive an intermediate by-product that can then undergo very efficient fermentation using methanogenic bacteria as above.

### II. Production of Memory Gem Using Carbon-Containing Gas

### EXAMPLE 5

### Diamond Film Deposition onto a Gem

Diamond-forming gas generally contains carbon and a carrier that are gaseous under the plasma conditions. Many appropriate carbon-containing substances, such as hydrocarbons, are known in the art. Organic compounds containing five or fewer carbon atoms are preferred due to their higher volatility, availability, and cost. Examples of hydrocarbons that are particularly useful are acetylene, ethylene, propane and methane, with methane being the most presently preferred. Carbon compounds that form C₂ radicals under plasma conditions are presently envisioned as being most useful. Additionally, the role of hydrogen gas may be of reduced significance in the deposition of diamond. Carbon-containing compounds that are more carbon rich, i.e. high carbon to hydrogen ratios, may prove very useful.

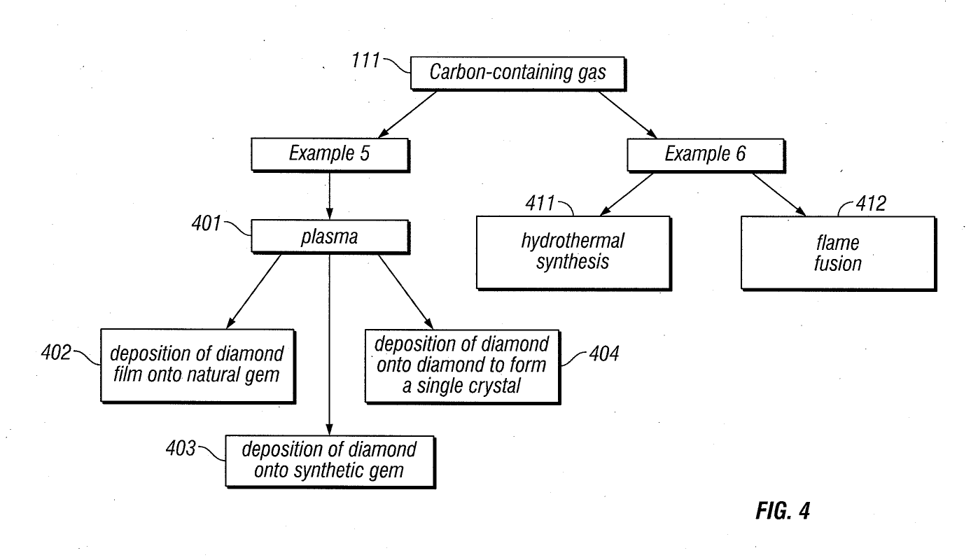

FIG. 4 illustrates several embodiments of the present invention using the process in Example 5 400. Carbon-containing gases 111 are used to produce a plasma 401 of a diamond-forming gas, which is deposed onto a substrate, preferentially composed of natural 402 and synthetic gems 403. Diamond coating and growth is achieved by CVD using these gaseous substrates. Examples of carbon-containing gaseous substrates such as hydrocarbons, carbon monoxide, methane, etc. are used for forming relatively pure diamond films, epitaxially and non-epitaxially.

A method can also be used for the production of diamond film, encompassing a substrate with plasma 401 under conditions such that the diamond film forms on the surface of the substrate, wherein the substrate is heated by the plasma.

In one embodiment, the diamond film is deposed onto a substrate that is a natural gem 402, such as natural diamond, ruby, sapphire, or other gem to produce a memory gem 121.

In another embodiment, the diamond film is deposed onto a substrate that is a synthetic gem 403, such as synthetic diamond produced by CVD or high-pressure high-temperature processes, or another synthetic gem, including but not limited to, synthetic diamonds, synthetic ruby or synthetic sapphires to produce a diamond-coated memory gem 121.

In yet another embodiment, the memory gem is produced from a homoepitaxial deposition of diamond onto a brilliant cut diamond anvil to produce a single crystal diamond 404 using a diamond anvil substrate, the substrate having a tip extending into the plasma, with a plasma 401 under conditions such that the monocrystalline diamond forms on the tip of the substrate 404.

### EXAMPLE 6

### Production of a Memory Gem with Carbon-Containing Gas Incorporated Throughout

Different processes for producing single crystal diamonds are illustrated in FIG. 4 under Example 6 410. For example, in one embodiment, carbon-containing gas 111 is incorporated into a synthetic gem during hydrothermal synthesis 411. This term encompasses various techniques for crystallizing substances at high temperatures and high pressures. Seed crystals are added to the solution, which grow into a single crystal.

In another embodiment, flame fusion 412 can be used to produce a crystal with carbon-containing gas 111 incorporated throughout. Carbon-containing gases 111 are combined with alumina or other substrates during flame fusion 412 to create synthetic gems. Specifically, aluminum oxide and chromium oxide are used to produce synthetic ruby. Aluminum oxide, titanium oxide, or ferrous iron oxide are used to produce synthetic sapphires.

As will be understood by those familiar with the art, the invention may be embodied in other specific forms without departing from the spirit or essential characteristics thereof. Likewise, the particular naming and division of the members, features, attributes, and other aspects are not mandatory or significant, and the mechanisms that implement the invention or its features may have different names, divisions and/or formats. Accordingly, the disclosure of the present invention is intended to be illustrative, but not limiting, of the scope of the invention, which is set forth in the following claims.

What is claimed is:

1. A method for producing a memory gem comprising:
fermenting a tissue to produce carbon-containing gases, wherein the tissue is one from the group consisting of animal tissue and human tissue, and wherein fermentation comprises hydrolysis for converting said tissue into byproducts;
methanogenesis for converting byproducts of said tissue into methane and carbon dioxide using a consortia of bacteria; and
deposing a diamond film onto a substrate through chemical vapor deposition.

2. The method of claim 1, wherein the substrate is a natural gem.

3. The method of claim 2, wherein the substrate is a diamond anvil, thereby producing a monocrystalline diamond.

4. The method of claim 1, wherein the substrate is a synthetic gem.

5. The method of claim 1, wherein said gem is one from the group consisting of:
diamonds, rubies, emeralds, sapphires, amethyst, opals, aquamarine, tanzanite, and alexandrite.

6. The method of claim 1, further comprising the steps of:
acidogenesis for converting said byproducts produced by hydrolysis into alcohols and fatty acids; and
acetogenesis for converting said fatty acids into acetic acid, hydrogen, and carbon dioxide, such that as a result of the combination of steps, digestion occurs.

7. The method of claim 1, wherein said bacteria used for methanogenesis contain at least one of engineered bacteria and natural isolated bacteria.

8. The method of claim 1, wherein the consortia of bacteria used during methanogenesis comprise at least one of Methanosarcina barkeri, Methanosaeta concilli, Methanobacterium ruminantium, M. formicium, M thermoautotrophicum, M arbophilicum, and M barkeri.

9. The method of claim 1, wherein the enzymes used during hydrolysis comprise at least one of keratinase, collagenase, elastase, endopeptidase, and proteinase.

10. The method of claim 1, further comprising the step of:
responsive to the presence of nitrogen before hydrolysis, denitrification for removing nitrogen.

11. The method of claim 1, wherein the tissue comprises at least one of hair, ash, protein, keratin, DNA, and RNA.

12. The method of claim 1, wherein the denitrification step occurs in an environment with a temperature of over 85 degrees Celsius with a pH of over 8.5.

13. The method of claim 12, wherein after denitrification ends, the pH is returned to 7.0 using a neutralizing buffer.

14. The method of claim 1, further comprising the step of protein degradation before hydrolysis using enzymes.

15. The method of claim 14, wherein the enzymes used during protein degradation comprise at least one of keratinase, collagenase, elastase, endopeptidase, and proteinase.

16. A method for producing a memory gem comprising:
fermenting a tissue to produce carbon-containing gases, wherein the tissue is one from the group consisting of animal tissue and human tissue, wherein fermentation comprises hydrolysis for converting said tissue into byproducts;
methanogenesis for converting byproducts of said tissue into methane and carbon dioxide using a consortia of bacteria; and
producing a synthetic gem using a process from the group consisting of flame fusion and hydrothermal synthesis by incorporating the carbon-containing gases into the mixture used for crystallization.

17. The method of claim 16, further comprising the step of:
responsive to the presence of nitrogen before hydrolysis, denitrification for removing nitrogen.

18. A memory gem produced by a method comprising:
fermenting a tissue to produce carbon-containing gases, wherein the tissue is one from the group consisting of animal tissue and human tissue, and wherein fermentation comprises hydrolysis for converting said tissue into byproducts;
methanogenesis for converting byproducts of said tissue into methane and carbon dioxide using a consortia of bacteria; and
deposing a diamond film onto a substrate through chemical vapor deposition.

19. The memory gem of claim 18 produced by a method further comprising:
acidogenesis for converting said byproducts produced by hydrolysis into alcohols and fatty acids; and
acetogenesis for converting said fatty acids into acetic acid, hydrogen, and carbon dioxide, such that as a result of the combination of steps, digestion occurs.

20. The memory gem of claim 18, produced by a method further comprising:
responsive to the presence of nitrogen before hydrolysis, denitrification for removing nitrogen.
